(19) United States
(12) Patent Application Publication
(10) Pub. No.: US 2004/0097960 A1
(43) Pub. Date: May 20, 2004

(54) Bag for laparoscopic surgery

(75) Inventors: Toshiro Terachi, Isehara (JP); Ryuichiro Niizeki, Kyoto (JP); Masaya Tatsumi, Kyoto (JP)

Correspondence Address: KODA & ANDROLIA, 2029 CENTURY PARK EAST, SUITE 1430, LOS ANGELES, CA, 90067-3024, US

(73) Assignee: J. Morita Manufacturing Corporation

(21) Appl. No.: 10/637,726

(22) Filed: Aug. 7, 2003

(30) Foreign Application Priority Data
Aug. 8, 2002 (JP) 2002-231224

(51) Int. Cl. A61B 17/26

(52) U.S. Cl. 606/114

(57) ABSTRACT

A bag for use in a laparoscopic surgery, having at its one end an opening through which extracted internal organ is put in and drawn from, and having at its other end a tubular organ storage portion with a closed bottom, of which bore diameter is smaller than that of the opening. The bag is characterized by being made of a soft synthetic resin, and its way part extending from the opening to the tubular organ storage portion being curved in a manner it intersects with the direction in which extracted internal organ is put in and drawn from at said opening.

### FIELD OF THE INVENTION

The present invention relates to a bag used for containing an extracted internal organ in an abdominal cavity or a thoracic cavity and for removing the contained internal organ out of a body cavity in a laparoscopic surgery.

### PRIOR ART

Laparoscopic surgery is an operation in an abdominal cavity or a thoracic cavity wherein treatment instruments such as a laparoscope, a cannula, an electric surgical knife and so on are inserted through an operation aperture which is cut to the minimum to observe the abdominal cavity with a laparoscope, to cut off a diseased internal organ, and to remove it out of the body cavity. This operation method has been widely applied because the operation aperture to be cut for operation is small so that a patient is recovered soon after the operation and invasion of bacteria is prevented to the minimum and further a patient's life quality after the operation is kept being improved.

In the operation, cut off internal organ is contained in a special bag in an abdominal cavity and is removed out of the body cavity with the bag so that adverse effects on other internal organs caused by scattering of the diseased tissue can be prevented promptly. Therefore, in principle, it is executed to remove the cut off internal organ contained in a bag regardless of benign or malignancy of the diseased tissue.

Such a bag is disclosed in JP-A-8-19544. The bag is made of a synthetic resin film and has a bag body having substantially the same diameter as its opening and a tail part narrowed from the bag body via a tapered part. In an actual operation, the bag is inserted in an operation site in an abdominal cavity to contain an extracted internal organ in its body and the bag is removed out of body cavity after closing the opening with a string. In case of removing, the bag is compressed by the pressure of the operation aperture wall and the extracted internal organ in the bag is smoothly moved to the narrowed tail part, thereby removing out of a body cavity through the operation aperture.

According to the above-mentioned bag, the extracted internal organ in the bag body is moved to the tail part to compress the extracted internal organ to be removed out of the body cavity so that the end-bag can smoothly pass through the operation aperture with narrow opening. However, because the bag body and the tail part are formed longitudinally and are made of a flexible synthetic resin film, there has been a problem such that the tail part is folded at the tapered part in an abdominal cavity, the extracted internal organ is stopped at the folded part so that it doesn't smoothly move to the tail part. Further, when the extracted internal organ is going to move to the tail part, air is collected at the bottom of the tail part to be prevented from escaping by the extracted internal organ so that the collected air keeps the extracted internal organ from moving to the tail part.

Thus if the bag is going to be removed out of the body cavity with the extracted internal organ not being moved to the tail part, the bag isn't smoothly removed and may damage the operation aperture wall because the part where the extracted internal organ is kept is stopped at the operation aperture.

### SUMMARY OF THE INVENTION

The present invention is proposed in order to solve the above-mentioned problems and provides a newly constructed bag for laparoscopic operation capable of drawing an extracted internal organ rapidly and smoothly.

(1) One embodiment of the present invention is a bag for use in a laparoscopic surgery having at its one end an opening through which extracted internal organ is put in and drawn from, and at its other end a tubular organ storage portion with a closed bottom, of which bore diameter is smaller than that of the opening, wherein the bag is made of a flexible soft synthetic resin, and wherein the way part extending from the opening to the tubular organ storage portion is curved so as to intersect with the direction in which the extracted internal organ is put in and drawn from at the opening.

The above-mentioned bag is put in an abdominal cavity or a thoracic cavity for a laparoscopic operation, an extracted internal organ such as cut off diseased tissue of adrenal, spleen, stomach, or gallbladder is contained in the bag from the opening by means of forceps to be drawn from the body cavity through the operation aperture cut for abdomen. In case of removal, the extracted internal organ in the bag is compressed by the operation aperture wall to move the extracted organ into the organ storage portion, because the way part extending to the organ storage portion is curved so as to intersect with the direction where the internal organ is put in or drawn from at the opening, there causes no folded part which prevents the extracted internal organ from moving in the abdominal cavity so that the extracted internal organ smoothly moves to the organ storage portion. Therefore, the extracted internal organ is contained in the organ storage portion with small diameter under compressed condition and passes through the small operation aperture without being stopped, resulting swift and smooth drawing from the body cavity.

(2) Other embodiment of the present invention is a bag for use in a laparoscopic surgery having at its one end an opening through which extracted internal organ is put in and drawn from, and at its other end a tubular organ storage portion with a closed bottom, of which bore diameter is smaller than that of the opening, wherein the bag is made of a flexible soft synthetic resin, and wherein a narrow tube made of soft material is provided within the bag in a manner that it is laid down from the opening to the tubular organ storage portion.

According to the above-mentioned construction, when the extracted internal organ is moved to the organ storage portion with small diameter, the air collected at the bottom of the organ storage portion is sequentially discharged to the opening via the narrow tube according to the movement of the extracted internal organ. Therefore, the air doesn't stay between the extracted internal organ and the bottom of the organ storage portion and the extracted internal organ is smoothly contained in the organ storage portion without causing air lock. Further, because the narrow tube is provided along the bag from the organ storage portion to the opening, the shape keeping function of the tube is added so that the bag isn't folded in an abdominal cavity to prevent the extracted internal organ from moving smoothly.

(3) According to further embodiment of the present invention, the above-mentioned embodiments (1) and (2) are combined. By the multiplier effects of both embodiments, the extracted internal organ is swiftly and smoothly removed out of body cavity and the embodiment is highly useful.

(4) According to other embodiment of the present invention, in the bag mentioned in the embodiment (1) or (3), the way part is formed in a curved line to be diagonal to the direction of putting and drawing the extracted organ in and from the opening. In this construction, the way part is formed so as to be curved in oblique direction against the putting-in or drawing-from direction of the extracted internal organ at the opening so that the extracted internal organ is smoothly contained in the organ storage portion without being stopped at the curved way part.

(5) According to other embodiment of the present invention, in the bag mentioned in the embodiment (1) or (3), the organ storage portion is formed so as to be curved at an angle equal to or above 90 degrees relative to the direction of putting and drawing the extracted internal organ in and from at the opening. In this construction, the organ storage portion is formed so as to be curved more than 90 degrees relative to the insertion and removal direction of the extracted internal organ through the opening so that the extracted internal organ or its diseased tissue contained in the organ storage portion is prevented from running out of or scattering from the bag because of the curved way part.

(6) According to other embodiment of the present invention, in the bag mentioned in the embodiment (2) or (3), the narrow tube is provided within the opening so as to be laid down along the edge of the opening. In this construction, the narrow tube is extended so as to be along the periphery of the opening so that the extended part forms like a ring and its shape is kept. When the bag is prepared to be put in an abdominal cavity, the opening is automatically widely opened, thereby facilitating easy insertion and removal of the extracted internal organ into or from the bag. Further the narrow tube is made of soft material, therefore, when the bag is put or drawn through the operation aperture, the opening is made compact by elastically deforming the narrow tube, thereby resulting smooth insertion and removal.

(7) According to other embodiment of the present invention, in the bag mentioned in any one of the embodiment (1)-(3), a string is provided at the periphery of the opening for closing the opening. In this construction, the extracted internal organ or its diseased tissue contained in the organ storage portion is prevented from being run or scattered out of the bag body by closing the opening with the string and further the opening is made compact, thereby resulting smooth removal of the extracted internal organ out of the body cavity.

(8) According to other embodiment of the present invention, in the bag mentioned in the embodiment (3), the narrow tube is provided along the larger side of the curved way part in its curvature. In this construction, by thus providing the narrow tube, the shape keeping effect by the tube is effectively achieved and further if the provided portion of the narrow tube is grabbed to be pulled out according to the curvature in case of removing the bag containing the extracted internal organ, it can be smoothly taken out.

### BRIEF DESCRIPTION OF THE DRAWINGS

FIG. 1 is an external view of the bag according to the first embodiment of the present invention.

FIG. 2 is an external view of the bag according to the second embodiment of the present invention.

FIG. 3 is an external view of the bag according to the third embodiment of the present invention.

FIG. 4a, FIG. 4b and FIG. 4c are conceptual views of the other embodiment of the present invention.

### DETAILED DESCRIPTION OF THE INVENTION

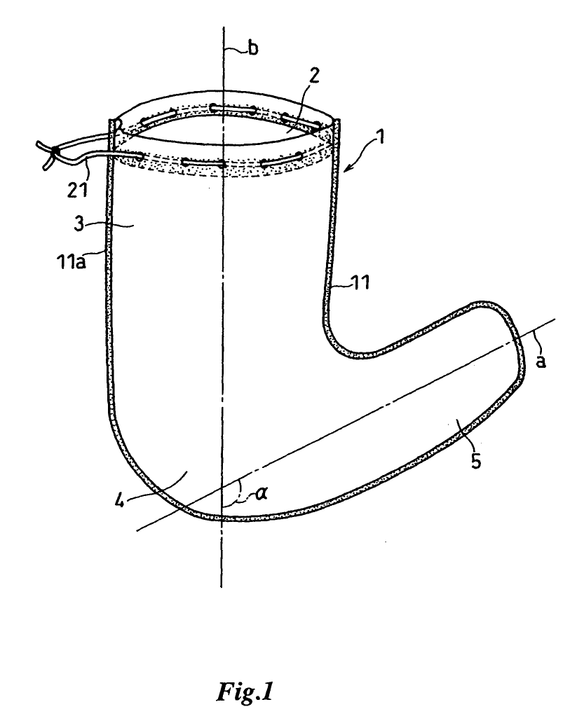

Now the embodiments of the present invention will be described referring to the attached drawings. The bag 1 shown in FIG. 1 is constructed such that two films made of polyurethane resin which are cut into the shape mentioned in the figure are overlapped and both peripheries except for its opening are deposited so as to form a bag with a bottom. The bag 1 is comprised of a bag body 3 with large diameter having an opening at one end and a tubular organ storage portion 5 with a bottom which has a small diameter and is extended from the other end of the body 3 via a curved way part 4.

String 21 is provided along the periphery of the opening 2 such that the opening 2 can be sealed with the string 21. The way part 4 is curved so as to be diagonal to the direction of the inserting or removing direction of the extracted internal organ through the opening 2. It is preferable that the longitudinal direction “a” has an angle α, which is more than 90 degrees, relative to the in-and-out direction “b” of the extracted internal organ at the opening 2.

In case of a laparoscopic operation using the above-mentioned bag 1, the bag 1 is put in through the operation aperture (not shown) cut open at an abdomen or a thorax to be positioned in the abdominal cavity or thoracic cavity (hereinafter they are called as abdominal cavity). If a trocar (guide cylinder) is provided for the operation aperture, the bag 1 is inserted in the abdominal cavity through the trocar or the operation aperture. The cut off diseased internal organ is contained in the body 3 of the bag 1 through the opening 2 by the operations of the forceps inserted through the operation aperture. The extracted internal organ is pushed forward as far as possible and the string 2 is pulled out to seal the opening 2.

After containing the extracted internal organ in the bag 1, one part of the string 21 (a knot in the figure) is pulled out of the operation aperture. Then the external deposited part 11a, which is a curved periphery of the body 3, is grabbed to pull out the bag 1 so as to rotate in a clockwise direction. When the extracted internal organ in the bag 1 reaches at the lower end of the operation aperture, the extracted internal organ in the body 3 is pushed into the organ storage portion 5 via the way part 4 to be contained. The extracted internal organ is contained in the organ storage portion 5 under a compressed condition so that the organ storage portion 5 passes through the small operation aperture and the bag 1 is entirely taken out of the body cavity smoothly.

In the above-mentioned extracting and removing operations of the diseased internal organ, because the bag 1 is comprised of the body 3, the curved way part 4 and the organ storage portion 5, the bag 1 isn't folded in the abdominal cavity so that the extracted organ isn't prevented from moving in the bag 1. Therefore, the extracted organ is smoothly moved from the body 3 to the organ storage portion 5 in case of removing out of the body cavity. Further, the curved angle is more than 90 degrees and the external deposited part 11a of the way part 4 is curved so that the bag 1 can be smoothly passed through the small operation aperture without uniformly being pulled out along the periphery of the opening 2 if the curved deposited part is gradually pulled out by grabbing with fingers or forceps.

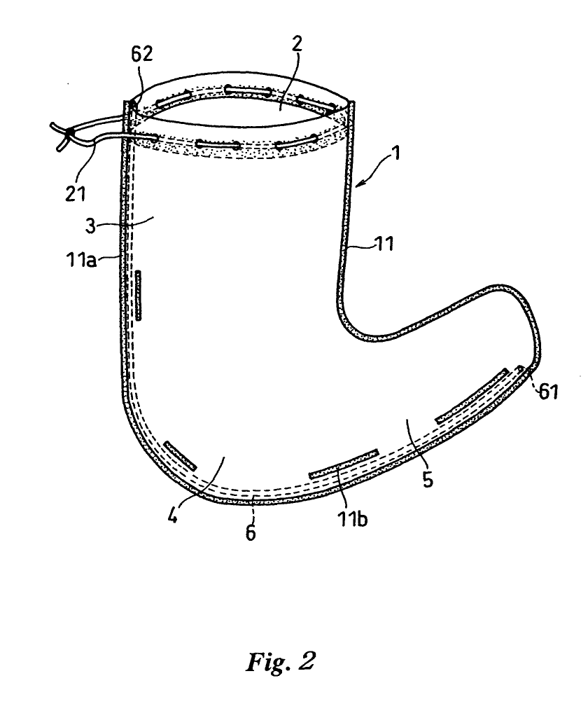

FIG. 2 shows a second embodiment of the present invention. The entire shape of the bag 1 is almost the same as that shown in FIG. 1, however, a soft narrow tube 6 made of polyurethane resin is provided along one side in the external deposited part 11a, which has a larger curvature of the way part 4. The tip end 61 of the tube 6 is provided around the bottom of the organ storage portion 5 and its terminal end 62 is extended to the opening 2. The reference 11b shows a deposited part for appropriately positioning the narrow tube 6. Other constructions are the same as those in FIG. 1 and the same reference numerals are allotted to omit their explanations.

For removing the diseased internal organ by means of the bag 1 of the second embodiment as mentioned in the first embodiment, when the extracted internal organ is moved from the body 3 to the organ storage portion 5, the air pushed into the bottom of the organ storage portion 5 by the extracted organ is sequentially discharged from the terminal end 62 through the tip end 61 and the tube 6. Therefore, the air doesn't stay between the extracted internal organ and the bottom of the organ storage portion 5 to prevent air lock so that the extracted internal organ is swiftly moved to the bottom of the organ storage portion 5.

According to the shape keeping effect of the narrow tube 6, the shape of the bag 1 is kept, thereby preventing the bag 1 from being folded in the abdominal cavity. Therefore, the extracted internal organ is surely and smoothly moved to the organ storage portion 5. Further, for drawing the bag 1 from the body cavity, if the part where the narrow tube 6 is provided is grabbed and gradually pulled out so as to rotate the bag 1 in a clockwise direction, the bag 1 is certainly taken out while keeping its shape.

Air communication space is formed between the deposited part 11b and the external deposited part 11a so that this space also has an air discharge function in addition to the narrow tube 6. It is preferable the deposited part 11b extends entirely from the bottom of the organ storage portion 5 to the opening 2 of the bag 1. In such a case the space formed by the deposited part 11b can be substituted for the air discharge means if the narrow tube 6 isn't provided.

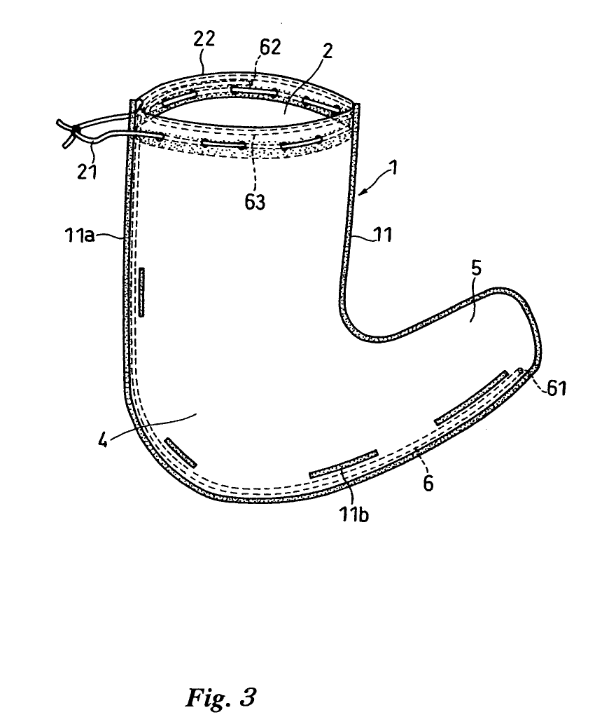

FIG. 3 shows a third embodiment of the present invention. The bag 1 of the present embodiment is almost the same as the one shown in FIG. 1 or FIG. 2, however, a narrow tube 6 is extended so as to form a ring 63 around the opening 2. The resin film forming the bag 1 is folded inwardly at the opening 2 so as to form like a sleeve 22 by deposition in order to be inserted with a ring like narrow tube 63 to be kept therein. The sleeve part 22 is provided with air through holes for communicating with outside. Other constructions of the bag 1 are the same as that shown in FIG. 1 or FIG. 2 and their explanations are omitted by allotting the same reference numerals.

When thus constructed bag 1 is put in an abdominal cavity as mentioned above, the narrow tube 6 is made of soft material and is elastically deformed so that the opening 2 is made compact to be passed through the narrow operation aperture. Further, when the bag 1 is put in the abdominal cavity, the ring-like part 63 is restored to be its original shape because of the elastic force of the narrow tube 6 and simultaneously the opening 2 is widely opened, thereby facilitating easy insertion of the extracted internal organ into the bag 1. For drawing the bag 1 containing the extracted organ from the body cavity, a string 21 is pulled out to elastically deform the ring-like part 63 and to seal the opening 2 as mentioned above. Further, the opening 2 is made compact and the bag 1 is easily taken out through the operation aperture.

In this embodiment, the ring-like part 63 is provided around the opening 2 as a part of the narrow tube 6 to achieve its shape keeping effect. However, if a soft ring-like member having shape memory function is provided for the opening 2 of the bag 1 shown in FIG. 1 or FIG. 2 other than the narrow tube 6, the same function can be brought out like the ring-like part 63 in case of putting in or drawn from the abdominal cavity. Therefore, such a member is preferably applied.

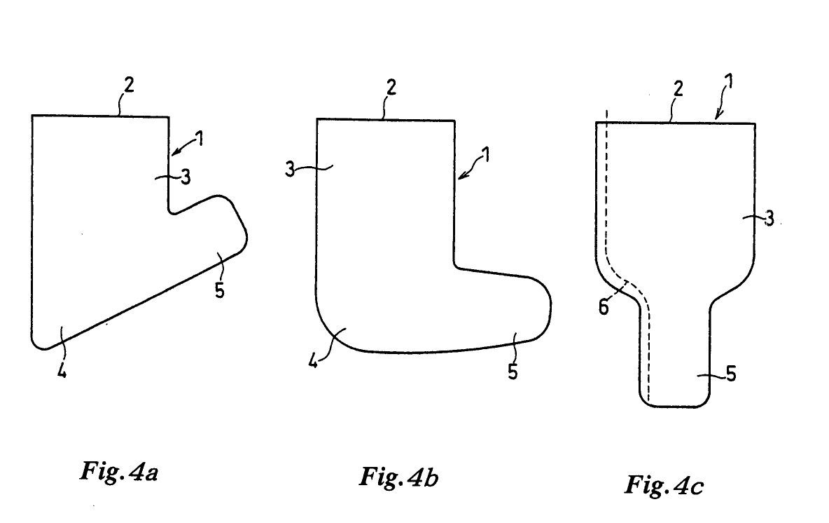

FIGS. 4a, 4b, 4c are conceptual views showing other embodiments of the present invention. The bags shown in FIG. 4a and FIG. 4b are modifications of the first embodiment. Only the shape from the body 3 to the organ storage portion 5 via the way part 4 is different from the first embodiment, and its basic technical idea is the same. Although, it isn't shown in the figures, it goes without saying that a string 21 may be provided, a narrow tube 6 may be provided therein, or other constructions mentioned in the second and the third embodiments may be added.

FIG. 4c is a modification of the second embodiment. The shape of the bag 1 in this modification is similar to the one disclosed in the prior art such that a tubular organ storage portion 5 with small diameter having a bottom is vertically extended from the body 3 and a narrow tube 6 is provided along the opening 2 to the bottom of the organ storage portion 5 in the bag 1.

Therefore, because of providing the narrow tube 6, it can be easily understood that air lock prevention function and the shape keeping function as mentioned in the second embodiment can be brought out according to thus shaped bag 1. Also in this embodiment, although it isn't shown in the figures, it goes without saying that a string 21 may be provided, a narrow tube 6 may be extended around the opening 2 to form the ring-like part 63, or other constructions mentioned in the second and the third embodiments may be added.

It goes without saying that any shape of the bag 1 other than the mentioned above can be adapted without departing from the spirit and the scope of the present invention. Further, the method for providing the narrow tube 6 may be modified appropriately other than the methods shown in the figures. Still further, soft synthetic resin with flexibility applicable for medical use can be used for the synthetic resin film made of the bag 1 other than the polyurethane resin.

1. A bag for use in a laparoscopic surgery having at its one end an opening through which extracted internal organ is put in and drawn from, and at its other end a tubular organ storage portion with a closed bottom, of which bore diameter is smaller than that of said opening,
wherein said bag is made of a flexible soft synthetic resin, and
wherein a way part extending from said opening to said tubular organ storage portion is curved so as to intersect with the direction in which said extracted internal organ is put in and drawn from at said opening.

2. A bag for use in a laparoscopic surgery having at its one end an opening through which extracted internal organ is put in and drawn from, and at its other end a tubular organ storage portion with a closed bottom, of which bore diameter is smaller than that of said opening,
wherein said bag is made of a flexible soft synthetic resin, and
wherein a narrow tube made of soft material is provided within said bag in a manner that it is laid down from said opening to said tubular organ storage portion.

3. A bag for use in a laparoscopic surgery having at its one end an opening through which extracted internal organ is put in and drawn from, and at its other end a tubular organ storage portion with a closed bottom, of which bore diameter is smaller than that of said opening,
wherein said bag is made of a flexible soft synthetic resin, and
wherein a way part extending from said opening to said tubular organ storage portion is curved so as to intersect with the direction in which said extracted internal organ is put in and drawn from at said opening, and further wherein a narrow tube made of soft material is provided within said bag in a manner that it is laid down from said opening to said tubular storage portion.

4. The bag for use in a laparoscopic surgery as set forth in claim 1 or 3, wherein said way part is formed in a curved line to be diagonal to the direction of putting and drawing the extracted organ in and from said opening.

5. The bag for use in a laparoscopic surgery as set forth in claim 1 or 3, wherein said organ storage portion is formed so as to be curved at an angle equal to or above 90 degrees relative to the direction of putting and drawing the extracted internal organ in and from at said opening.

6. The bag as set forth in claim 2 or 3, wherein said narrow tube is provided within said opening so as to be laid down along the edge of said opening.

7. The bag as set forth in any one of claims 1 to 3, wherein a string is provided at the periphery of said opening for closing said opening.

8. The bag as set forth in claim 3, wherein said narrow tube is provided along the larger side of said curved way part in its curvature.
